(19) United States
(12) Patent Application Publication
(10) Pub. No.: US 2024/0115844 A1
(43) Pub. Date: Apr. 11, 2024

(54) MEDICAL PARTICLE STORAGE TUBE, DRUG LOADING METHOD, AND METHOD FOR DELIVERING DRUG-LOADED MICROPARTICLES

(71) Applicant: SUZHOU YIBEN LIFE TECHNOLOGY CO., LTD, Suzhou (CN)

(72) Inventors: Shaoge GU, Suzhou (CN); Yonghua DONG, Suzhou (CN); Lei JIN, Suzhou (CN); Liangliang ZHAO, Suzhou (CN); Guoxiang SUN, Suzhou (CN)

(73) Assignee: SUZHOU YIBEN LIFE TECHNOLOGY CO., LTD, Suzhou, JS (CN)

(21) Appl. No.: 18/542,759

(22) Filed: Dec. 17, 2023

Related U.S. Application Data
(63) Continuation of application No. PCT/CN2022/099565, filed on Jun. 17, 2022.

(30) Foreign Application Priority Data
Jun. 17, 2021 (CN) 202121349663.6

(51) Int. Cl. A61M 37/00

(52) U.S. Cl. CPC A61M 37/0069; A61M 2202/0007; A61M 2202/06

(57) ABSTRACT

A particle storage tube (100) used for implanting microparticles into a body in cooperation with a puncture needle (500), and comprising an accommodation portion (1) and a drug delivery portion (2). The accommodation portion (1) is an elongated hollow tube having a channel (10) used to accommodate microparticles (4). One end of the accommodation portion (1) is connected to the drug delivery portion (2), and the other end of the accommodation portion (1) is an open part (11) used to discharge the microparticles out of the particle storage tube (100). The inner diameter of the channel (10) is 1.0-1.8 times the inner diameter of a lumen (502) of the puncture needle (500). An inner chamber (20) of the drug delivery portion (2) is in communication with the channel (10), and is used for delivering liquid medicine (200) to the channel (10).

### BACKGROUND

### Technical Field

The present invention relates to a medical micro-particles storage tube for pharmaceutical use, and also relates to a drug loading method using the medical micro-particles storage tube and a delivering method of drug-loadable micro-particles, belonging to the technical field of pharmaceutical.

### Related Art

In prior Chinese patent application No. CN111467319A, a drug loadable micro-particles is disclosed, including a shell and a drug loading part inside the shell, which is used to be implanted into the body tissues through a puncture needle. The shell has at least one micro-hole through the wall of the shell, and the drug loaded part is of a material adapted to carry drugs. The drug loadable micro-particles can load different kinds of drugs, and can be directly implanted into tissues, which has the technical advantages of microspheres and radioactive micro-particles. In addition, different micro-particles perform different drug release velocity curves, so as to achieve precise control of drug release. Further, by implanting micro-particles with different drugs at one time, different drugs can promote each other and improve the efficacy.

However, because the micro-particles themselves are small, and the micro-holes on the shell are of only micron size, thus, it is difficult for drug liquids, emulsions and suspensions to enter the shells under the effect of surface tension. Therefore, too little drug can be loaded by the micro-particles, which may lead to failure to achieve the expected medical effect. On the other hand, for millimeter sized micro-particles to be implanted into the organism, special delivery tools are needed to ensure delivering the micro-particles to the target location. Therefore, there is a need for auxiliary tools specially for loading the drugs to the micro-particles.

### SUMMARY

The primary technical problem to be solved by the invention is to provide a medical micro-particles storage tube;

A second technical problem to be solved by the invention is to provide a drug loading method using the medical micro-particles storage tube.

A third technical problem to be solved by the invention is to provide a method for delivering drug-loadable micro-particles using the medical micro-particles storage tube.

In order to achieve the above technical purpose, the invention adopts the following technical scheme.

According to the first aspect of the embodiment of the present invention, here is provided a medical micro-particles storage tube for implanting micro-particles into a body in cooperation with a puncture needle, the medical micro-particles storage tube comprising a holding part and a drug delivery part, wherein
the holding part is an elongated hollow tube with a channel for accommodating the micro-particles; one end of the holding part is connected with the drug delivery part, and the other end of the holding part is an opening part for discharging the micro-particles out of the medical micro-particles storage tube,
an inner diameter of the channel is 1.0-1.8 times the inner diameter of a needle path of the puncture needle;
an inner cavity of the drug delivery part is connected with the channel for conveying drug solution to the channel.

Preferably, the drug delivery part includes a liquid collecting structure for accumulating the drug solution, and the liquid collecting structure is configured to: 1) accumulate the drug solution when no air exhaust; 2) ensure the air will not pass through the drug solution when the air flows from the opening part to the drug delivery part along the channel; 3) allow the drug solution can enter the channel when the air flows from the drug delivery part to the opening part.

Preferably, the drug delivery part comprises a collecting part and a liquid collecting part,
one end of the liquid collecting part is connected with the collecting part, and the other end is connected with the channel for storing the drug collecting liquid.

Preferably, the liquid collecting part has a shape configured to meet the following requirements:
when the liquid collecting part is in a liquid delivery state, the lowest part of the liquid collecting part is located on the axis of the channel as the lowest position of the liquid delivery;
when the liquid collecting part is in a liquid collecting state, the lowest part of the liquid collecting part is far away from the channel as the lowest position of the liquid collecting part;
the lowest position of the liquid collecting part is different from the lowest position of the liquid delivery.

Preferably, a support frame fixed to or connected with a periphery of the drug delivery part to maintain the medical micro-particles storage tube in an inclined state.

Preferably, the opening part is configured to conform to Luer interface technical standard.

Preferably, a plug is detachably installed on the opening part for plugging the opening parts.

Preferably, the drug delivery part comprises an outer wall and an inner wall, the inner wall forms an air cavity, and the air cavity is aligned with the channel to form an air path;
the inner wall is connected with the outer wall to form a drug solution cavity separated from the air cavity;
the drug solution cavity is communicated with the air cavity through a through hole.

Preferably, a plurality of drug solution cavities is formed between the inner wall and the outer wall.

Preferably, the through hole comprises an air end connecting the air cavity and a drug solution end connecting the drug solution cavity,

The air end is closer to the channel than the liquid end.

Preferably, the through hole has a diameter that ranges from 0.1 to 0.4 mm.

Preferably, the drug delivery part comprises a collecting part and a transition part; one end of the transition part is connected to the channel, and the other end is connected to the collecting part, and the inner diameter of the transition part increases gradually from a diameter that is the same as that of the channel, to a diameter that is the same as that of the collecting part.

Preferably, the drug delivery part includes a collecting part, a transition part and a gathering part, and the gathering part connects the collecting part and the transition part;
the collecting part has an inner diameter which increases from a diameter that is the same as that of the transition part, gradually to a diameter that is the same as that of the collecting part;
the transition part has an inner diameter which increases from a diameter that is the same as that of the channel, gradually to a diameter that is the same as that of the gathering part.

According to the second aspect of the embodiment of the present invention, a drug loading method using the aforementioned medical micro-particles storage tube, comprising the following steps:
micro-particles are inserted into a channel in the medical micro-particles storage tube;
drug solution is injected into a drug delivery part of the medical micro-particles storage tube;
negative pressure is applied to the medical micro-particles storage tube until no air escapes, or apply negative pressure for a fixed duration, with a negative pressure that allows the air to be pumped out without the drug solution;
the negative pressure is stopped, or positive pressure is applied, and then the drug solution is injected into the channel until the drug solution stops flowing.

According to the third aspect of the embodiment of the present invention, a method for delivering drug loadable micro-particles using the medical micro-particles storage tube, comprising the following steps:
micro-particles are inserted into a channel of the medical micro-particles storage tube;
an open part of the medical micro-particles storage tube is aligned with a puncture needle;
a push rod is pushed into the channel, and the micro-particles are pushed out of the channel.

Compared with the prior art, the invention has the following technical effects:
1) using pressure difference, the drug solution is able to overcome the factors such as surface tension, to enter the small micro-particles and fully fill the micro-particles;
2) a two-stage or three-stage structure is used to solve the present problem that the drug solution cannot enter or completely fill the interior of the micro-particles in the elongated holding part when loading drugs. Therefore, the medical micro-particles storage tube provided by the invention can increase the drug loading capacity of the micro-particles, and the equipment required for drug loading is simple, the drug loading speed is fast, and the operation is simple;
3) in implanting, the medical micro-particles storage tube provided by the invention can be used in combination with a conventional puncture needle through a Luer connector, so the operation of micro-particles implantation is simple and conformable to the doctors; and
4) the medical micro-particles storage tube provided by the invention has simple sterilization operation, convenient installation and use, and is suitable for transportation and storage under normal temperature and pressure for a long time.

### BRIEF DESCRIPTION OF THE DRAWINGS

FIG. 1 shows a sectional structure of a medical micro-particles storage tube in the first embodiment of the present invention;

FIG. 2 is a side view of the medical micro-particles storage tube in the first embodiment of the present invention;

FIG. 3 is a schematic diagram of an assembly of the medical micro-particles storage tube in the first embodiment of the present invention;

FIG. 4 is a schematic diagram of a drug-loadable micro-particles in the embodiments of the invention;

FIG. 5A is a partial view of the medical micro-particles storage tube in a second embodiment of the present invention;

FIG. 5B is a schematic diagram of a sectional view of the medical micro-particles storage tube in FIG. 5A;

FIG. 6A is a schematic exploded view of the medical micro-particles storage tube in a second embodiment of the present invention;

FIG. 6B is a front view of the medical micro-particles storage tube in FIG. 6A;

FIG. 7A is a schematic cross-sectional view of a medical micro-particles storage tube in the third embodiment of the present invention;

FIG. 7B is a partial enlarged view of a dotted circle in FIG. 7A;

FIG. 7C is a schematic top view of a drug delivery part in FIG. 7A;

FIG. 7D is a schematic top view of another drug delivery part in FIG. 7A;

FIG. 7E is a schematic three-dimensional view of a medical micro-particles storage tube in the third embodiment of the present invention;

FIG. 8A is a schematic three-dimensional view of a medical micro-particles storage tube in the fourth embodiment of the present invention;

FIG. 8B is a schematic diagram of the internal structure of the medical micro-particles storage tube in the fourth embodiment of the present invention;

FIG. 8C is a schematic diagram of a liquid collection step using the medical micro-particles storage tube shown in FIG. 8B;

FIG. 8D is a schematic diagram of a step of implementing negative pressure to the medical micro-particles storage tube shown in FIG. 8C;

FIG. 8E is a schematic diagram of a liquid loading step of the medical micro-particles storage tube shown in FIG. 8D;

FIG. 8F is a schematic diagram of a step of pressurizing liquid into the medical micro-particles storage tube shown in FIG. 8E;

FIG. 9 is a schematic diagram of inserting a puncture needle into the medical micro-particles storage tube shown in FIG. 8A.

### DETAILED DESCRIPTION

The technical content of the present invention will be described in detail below in combination with the accompanying drawings and specific embodiments. For the convenience of understanding and description, the right side of the drawing is referred to as “the right side”, and the left side of the drawing is referred to as “the left side”, but this does not constitute a limitation or constraint to the present invention.

The medical micro-particles storage tube provided by the embodiment of the invention is used for loading and delivering drugs by micro-particles with micropores, so it can also be called a medical micro-particles storage tube. With negative pressure drug loading technology, the medical micro-particles storage tube can improve the capacity of the drug solution entering the micropores, so as to increase the drug loading capacity of each micro-particle. Further, the isolation of air path and liquid path within the medical micro-particles storage tube avoids the impact of liquid splash caused by air escape on the drug loading. Therefore, using the medical micro-particles storage tube provided by the embodiment of the invention to load drug in the micro-particles, the drug loading capacity (measured by the liquid volume in micro-particles/the volume of micro-particles) can reach 50-94%.

Moreover, the medical micro-particles storage tube provided by the embodiment of the present invention, when playing the role of micro-particles storage, provides constraints on the relative position between micro-particles, so that each micro-particles is arranged in a string, and the relative position therebetween will not change. When the medical micro-particles storage tube perform as a tool for micro-particles delivery, during implantation, the micro-particles are pushed into the tissue in a line along the channel of the medical micro-particles storage tube, so that the micro-particles are arranged in sequence (there can be a gap or no gap between two adjacent micro-particles) and remain in the tissue. Therefore, it is conducive to the synergistic effect of different drugs in micro-particles. Specifically, the micro-particles loaded with different drugs implanted at the same time can make different drugs sustained and/or controlled release in the tissue simultaneously, thus forming a synergistic effect. Further, the micro-particles arranged in strings can play a special synergistic effect for their positional relationship among micro-particles (the micro-particles each can remain in the tissue), especially the micro-particle arranged in front and the micro-particle arranged in the rear (for example, the micro-particles with developer arranged in front are used for contrast; the micro-particles with hemostatic agent arranged in rear are used for needle tract closure).

Therefore, with the medical micro-particles storage tube provided by the embodiment of the invention, it can not only implant micro-particles that are loaded with different drugs at the same time, but also fix the micro-particles within a target position without any movement. The movement to be avoid includes the micro-particles' removing in the tissue and the relative movement between neighboring micro-particles. Thus, a plurality micro-particles can be settled at different positions by one-time implanting with the medical micro-particles storage tube, which enable the micro-particles individually to release different drugs at different positions along the puncture needle path (needle tract) within the tissue at different release times (in a slow and controlled release manner). That is impossible for dosage such as microspheres that cannot be accurately positioned in the tissue.

### First Embodiment

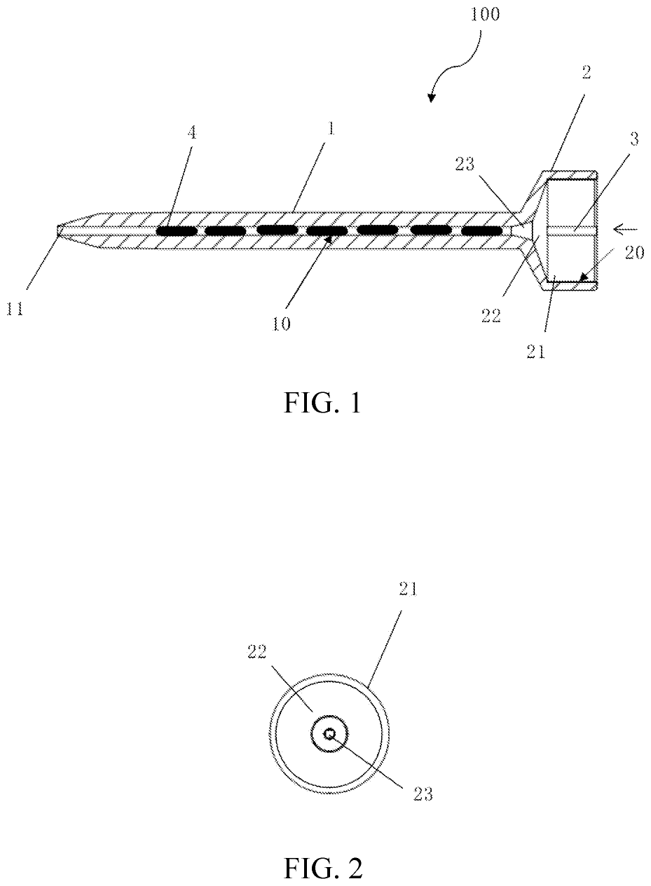

As shown in FIG. 1, the first embodiment of the present invention provides a medical micro-particles storage tube 100 for delivering drug loadable micro-particles or radioactive seeds (hereinafter collectively referred to as micro-particles) to the body. The medical micro-particles storage tube 100 includes a holding part 1 and a drug delivery part 2. Preferably, the medical micro-particles storage tube 100 further includes a plug 5 and a cover 6.

Specifically, the holding part 1 is connected with the drug delivery part 2. The holding part 1 is an elongated hollow tube. A left end of the drug delivery part 2 connects with one end (a right end in FIG. 1.) of the holding part 1. An opening part 11 is located at the other end (a left end in FIG. 1) for discharging micro-particles from the medical micro-particles storage tube 100. The holding part 1 has a channel 10 extending along an axis of the hollow tube. The inner diameter of the channel 10 is 0.5-1.8 mm, which matches the size of the drug-loadable micro-particles and is compared to the inner diameter of the needle path of the puncture needle (i.e., 1.0-1.8 times the inner diameter of the needle path, and preferably, 1.0-1.3 times), so that the micro-particles 4 can not only be relatively fixed within the channel 10 by friction, but also be allowed to move along the channel 10 to enter the organism/tissues via injection. Moreover, the volume within the channel 10 should be as small as possible to reduce the waste of drug solution. For example, if the inner diameter of the puncture needle is 0.6 mm, the inner diameter of the channel 10 is 0.6-1 mm; If the inner diameter of the puncture needle is 0.7 mm, the inner diameter of the channel 10 is 0.71.2 mm; if the inner diameter of the puncture needle is 0.8 mm, the inner diameter of the channel 10 is 0.81.4 mm. It can be understood that the inner diameter of the channel 10 can also be designed to be 1 mm, so that the medical micro-particles storage tube can be used separately with three specifications of puncture needles those are of inner diameters of 0.60.8 mm. At one end of the holding part 1, close to the opening part 11, the outer diameter of the holding part 1 gradually becomes thinner and thinner.

In case of using the medical micro-particles storage tube 100 provided by the embodiment of the present invention, a plurality of micro-particles 4 are placed inside the holding part 1, and the micro-particles 4 that have been loaded drug solution are implanted into the body through the opening part 11. In this example, the drug loading capacity of the entire medical micro-particles storage tube (the total drug capacity in the multiple micro-particles) is 0.04 ml, less than 1 ml. It is because the drug capacity is very small, for the surface tension of the liquid, the micropore size on the micro-particles shell is too small and other factors, the conventional method cannot be used to load drug in the micro-particles rapidly and effectively. So, the drug loading needs to be carried out by using the medical micro-particles storage tube. The number and type of micro-particles 4 can be designed according to the actual needs.

The drug delivery part 2 is in a funnel shape as a whole, and its inner cavity 20 is connected with the channel 10 to transmit the drug solution to the channel 10. From right to left in FIG. 1, it successively includes a cylindrical collecting part 21, a cone-shaped gathering part 22 and a frustum-of-cone shaped transition part 23. As shown in FIG. 2, the left view of the drug delivery part 2 (viewed from the direction of the arrow in FIG. 1) shows three concentric circles, and from the outside to the inside are the cylindrical collecting part 21, the frustum shaped gathering part 22 and the frustum-of-cone shaped transition part 23. In practice, the drug solution is injected in the drug delivery part 2, and then enters the interior of the holding part 1 through the collecting part 21, the gathering part 22 and the transition part 23, so that the drug solution can be loaded in the micro-particles 4 in the holding part 1.

As shown in FIG. 1 and FIG. 2, the inner diameter of the cylindrical collecting part 21 is obviously larger than that of the accommodation part 1, for example, the inner diameter of the collecting part 21 is larger than 3 times of the inner diameter of the accommodation part 1. In this embodiment, in order to collect more drug solution, the inner diameter of the collecting part 21 is more than 5 times the inner diameter of the channel 10 of the holding part 1, preferably more than 10 times.

The collecting part 21 is connected with the cone-shaped gathering part 22, and is arranged with a plurality of ventilation holes 3 on its inner surface. The inner diameter (first inner diameter) at where the gathering part 22 connects with the collecting part 21 is D1, the inner diameter (second inner diameter) at where the gathering part 22 connects with the transition part 23 is D2, the inner diameter at where the transition part 23 connects with the holding part 1 is D3. The three inner diameters meet the conditions of D1>D2>D3. In this embodiment, D2 is 1.6-3 times of D1, preferably 2.2 times; D3 is more than 8 times of D2, preferably 10-15 times, and preferably 12 times. With different three inner diameters, it is verified by experiments that the drug loading speed is fast and the drug loading amount in micro-particles 4 is large.

The transition part 23 makes the inner diameters increase gradually from D1 to D2, so that the air in the holding part 1 can slowly gather together along the transition part 23, then escape from the gathering part 22 to the collecting part 21, and finally discharge to the outside of the micro-particles storage pipe. At the same time, the drug solution enters the transition part 23 along the gathering part 22 from the collecting part 21, and then further accumulates into the inner cavity of the holding part 1. When the air discharged from the drug solution, it will not break bubbles or gather bubbles, so it will be discharged out of the medical micro-particles storage tube in the form of tiny bubbles. It avoids the phenomenon of forming large bubbles due to the accumulation of bubbles, which causes the liquid barrier. It is also avoid that the bullae prevents the drug solution to enter the inner cavity of the holding part; and that the amount of drug loaded is insufficient (there is not enough drug solution to enter the micro-particles). The medical micro-particles storage tube 100 provided by the embodiment of the present invention can prevent the liquid barrier phenomenon after the drug solution enters the holding part 1, due to the drug delivery part 2 with the above structure. The liquid barrier phenomenon refers to the phenomenon that liquids are separated from each other in an elongated space, making the liquid become separated droplets.

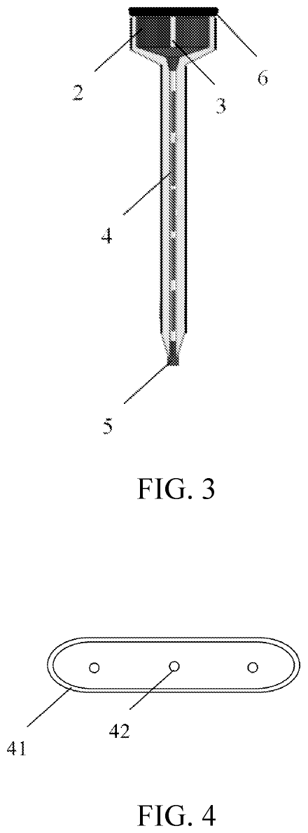

FIG. 3 provides a schematic diagram of the overall installation of the medical micro-particles storage tube 100. As shown in FIG. 3, the cover 6 is T-shaped and is installed to the drug delivery part 2. The plug 5 is also T-shaped and is installed at the end of the holding part 1 away from the drug delivery part 2. Both make the holding part 1 and the drug delivery part 2 form a closed space. The cover 6 is provided with micro holes (not shown) and is connected with the ventilation tank 3 through the collecting part 21, so that during sterilization operation, ethylene oxide air can pass through the micro holes through the cover 6, flow through the ventilation tank 3 and enter the holding chamber 1.

For the convenience of understanding, the structure of micro-particles 4 is shown in FIG. 4, and a plurality of drug feeding holes 42 are provided on the closed housing 41. The size of the dosing hole 42 should meet the following requirements: 1) allowing the drug solution to enter the housing 41 under a certain air pressure; 2) preventing the drug solution from flowing out of the shell 41 under normal pressure after entering the shell 41; 3) not reducing mechanical strength or structural shape of the housing 41.

### Second Embodiment

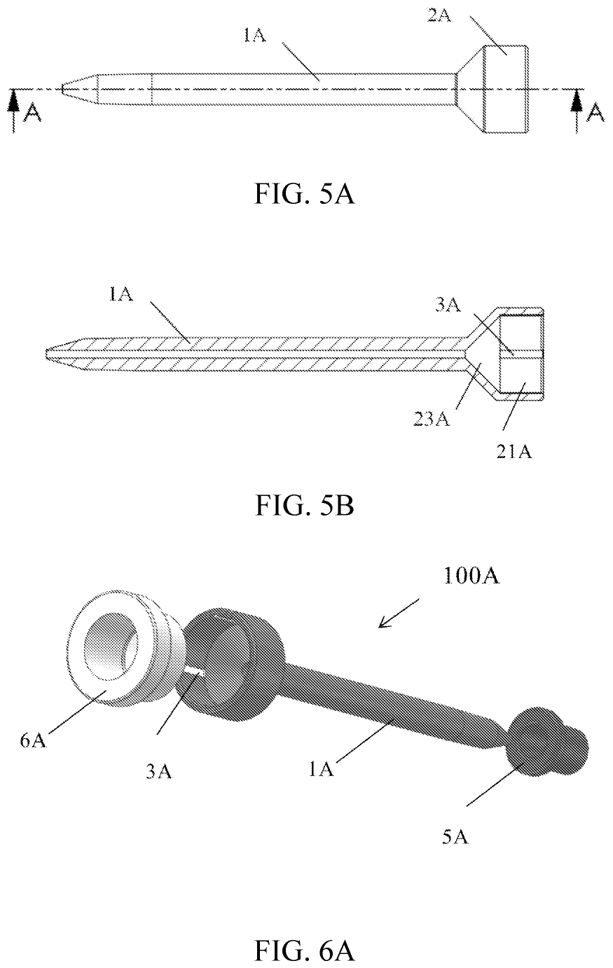

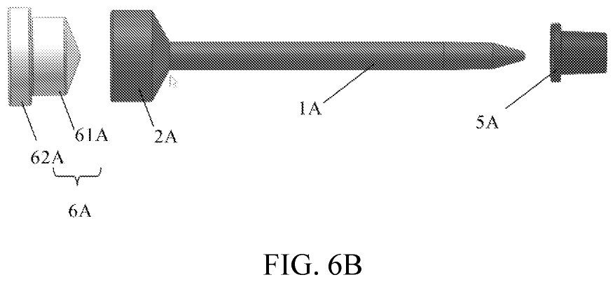

FIG. 5A, FIG. 5B, FIG. 6A and FIG. 6B provide another form of medical micro-particles storage tube, including the holding part 1A, the drug delivery part 2A, the plug 5A and the cover 6A.

The drug delivery part 2A connected with the holding part 1A in this embodiment is a two-stage structure. That is, only the collecting part 21A and the transition part 23A. The collecting part 21A may be the same as the collecting part 21 of the first embodiment, and is provided with a ventilation hole 3A. The collecting part 21A can also be different from the collecting part 21 (that is, there is no ventilation hole).

Compared with the transition part 23 of the first embodiment, the transition part 23A in this embodiment has a larger length in the longitudinal direction of the medical micro-particles storage tube. One end of the transition part 23A is connected with the holding part 1A (whose inner diameter is DD. The other end of the transition part 23A is connected with the collecting part 21A (whose inner diameter is D3). As the length of the transition part 23A increases, its shape is able to vary sufficiently gradually so that the air coming out of the inner cavity of the holding part 1A can travel slowly along the transition part 23A without agglomeration due to the rapid expansion of the space, thus avoiding the formation of large bubbles.

The plug 5A in this embodiment is used to seal one end of the holding part 1A from outside, which is different from the plug inserted the interior of the holding part 1 in the first embodiment. The cover 6A includes an inner body 61A and an outer body 62A. Among them, the shape of the inner body 61A is compatible with the shape of the collecting part 21A and the transition part 23A to match well and achieve the sealing effect. The outer diameter of the outer body 62A is larger than that of the inner body 61A, and micro-holes can be formed at the outer edge to communicate with the ventilation hole 3A arranged in the collecting part 21A to pass gases such as ethylene oxide for sterilization operation.

The medical micro-particles storage tube provided by the second embodiment of the invention can feed micro-particles into a conventional puncture needle, as long as the size of the holding part is compatible with the puncture needle. For example, the puncture needle and the holding part are both of 18F. The micro-particles are also of size corresponding to the drug delivery part 2 can be contained in the medical micro-particles storage tube for delivery and storage. When in use, pull out the plug, insert the Luer connector, and then connect it to the puncture needle through the Luer connector. Then, open the cover, put in the push rod, and push the micro-particles from the holding part through the Luer connector into the puncture needle. Finally, the micro-particles were implanted into the body by using the conventional operation of puncture needle.

Whether the three-stage structure in the first embodiment or the two-stage structure in the second embodiment of the invention is adopted, the pipe diameter design of the drug delivery part needs to comprehensively consider the viscosity of the drug solution, the loading amount of the drug solution, the air pressure used to load the drug solution, the size of the first inner diameter D1, the loading time and other factors. Therefore, the value in this embodiment is adjustable according to practice needs. In order that the viscosity needs be larger, the loading amount of drug solution needs be larger, or the loading time needs to be shortened, the measures can be take that the air pressure can be increased while the speed of vacuum (negative pressure) is accelerated, or D2 or D3 becomes larger while the transition part becomes longer (the pipe diameter changes evenly and gradually).

When it needs to be explained, the medical micro-particles storage tube provided by the embodiment of the invention can be used not only in the post-drug-loading mode that the micro-particles without drug are put into the holding part of the medical micro-particles storage tube and then the micro-particles are loaded with drugs, but also in pre-drug-loading mode that the micro-particles are loaded with drug in advance and then they are directly put into the holding part of the medical micro-particles storage tube for storage.

Due to the convenience of drug loading, doctors can configure different drug solutions which are loaded into multiple micro-particles according to the practice before surgery, and then implant them into the human body respectively to improve the curative effect. For example, the first drug solution is loaded into some micro-particles, then the second drug solution is loaded into others, and finally these two kinds of micro-particles are implanted into the human body respectively.

To sum up, the medical micro-particles storage tube provided by the embodiment of the invention uses a two-stage or three-stage structure to solve the problem that the drug solution cannot enter or completely fill the micro-particles inside the elongated delivery part when loading drugs. Therefore, the medical micro-particles storage tube provided by the embodiment of the invention can increase the drug loading capacity of micro-particles, and the equipment required for drug loading is simple, the drug loading speed is fast, and the operation is simple. Secondly, when in use, the medical micro-particles storage tube provided by the embodiment of the invention can be combined with the conventional puncture needle through the Luer connector, so the operation of micro-particles implantation is simple and in line with the habits of doctors. Moreover, the medical micro-particles storage tube provided by the embodiment of the invention has simple sterilization operation, convenient installation and use, and is suitable for long-time transportation and storage under normal temperature and pressure.

### Third Embodiment

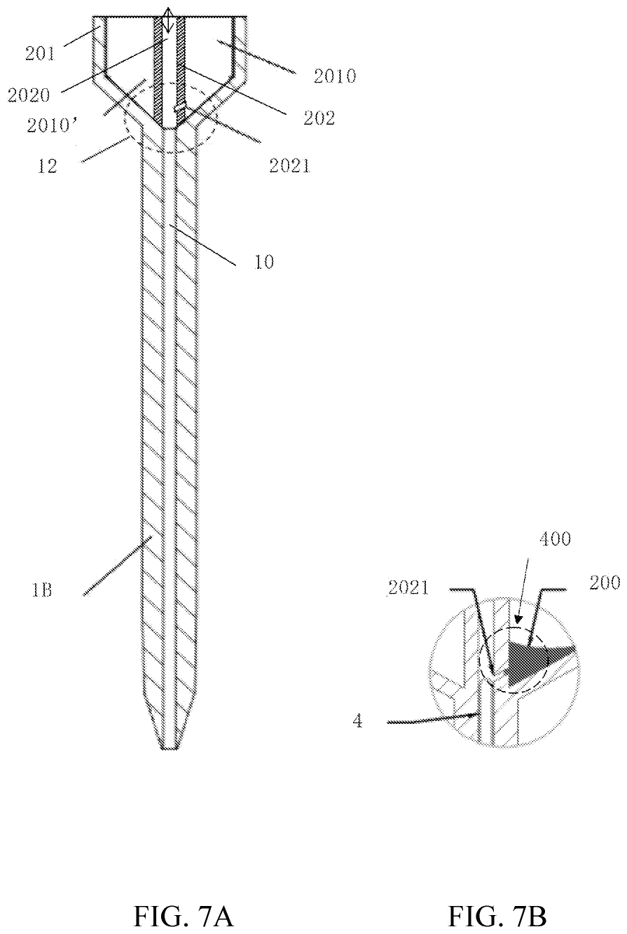

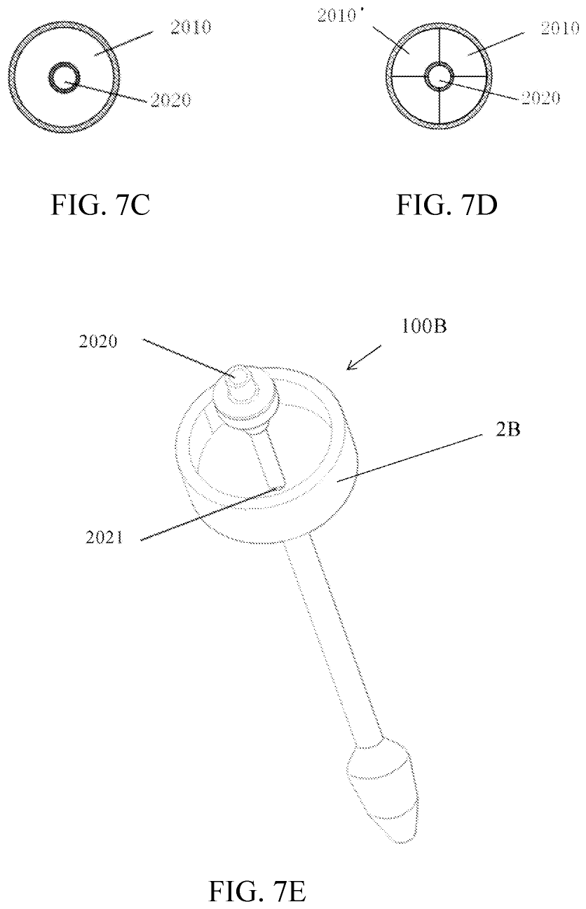

As shown in FIG. 7A to FIG. 7E, the third embodiment of the present invention provides a medical micro-particles storage tube 100B, including a holding part 1B and a drug delivery part 2B. By using the drug loading device, loading the drug solution 200 into the micro-particles in the medical micro-particles storage tube 100b, the micro-particles 4 in the medical micro-particles storage tube can be pushed into the puncture needle for implantation or directly implanted into the body.

In this embodiment, the drug delivery part 2 includes a liquid collecting structure 400 for storing the drug solution 200. The liquid collecting structure 400 is designed to meet the following three conditions: 1) the liquid collecting structure 400 can store the drug solution 200 without air exhaust; when the air flows from the opening part 11 to the drug delivery part 2 along the channel 10 (i.e. air exhaust), that is, the air goes up in FIG. 7A, it will not pass through the drug solution 200; and 3) when the air flows from the drug delivery part 2 to the opening part 11 (i.e. air intake), the drug solution can enter the channel 10.

In this embodiment, the liquid collecting structure 400 includes an inner wall 202, a drug solution cavity 2010, and a through hole 2021.

In this embodiment, the holding part 1B is roughly the same as the holding part 1 in the first embodiment. It is a tube shape with a channel 10 extending along the axis of the tube for delivering the micro-particles 4.

The drug delivery part 2B in this embodiment includes an outer wall 201 and an inner wall 202. In this embodiment, the inner wall 202 is tubular. The inner wall 202 has an inner cavity (the air cavity 2020) aligned with the channel 10 of the holding part 1B, and its inner diameter is the same or slightly larger than the inner diameter of the channel 10, for example, 1.0-1.2 times. Therefore, the air cavity 2020 surrounded by the inner wall 202 penetrates with the channel 10 to form an air path (as shown by the arrow in FIG. 7A).

The inner wall 202 is connected with the outer wall 201 and the holding part 1B, and thus provides a neck 12 (the area shown by the dotted circle in FIG. 7A) at the connection. The neck 12 is also an end of the holding part 1B, which is far away from the opening part 11. The inner wall 202 is connected with the outer wall 201 at the neck 12 (for example, they are integrally formed), thereby forming the air cavity 2020 and the drug solution cavity 2010 isolated with each. In this embodiment, the drug solution cavity 2010 has an annular space surrounding the inner cavity 2020 (as shown in FIG. 7C).

It can be understood that as shown in FIG. 7B, FIG. 7C and FIG. 7D, the space between the inner wall 202 and the outer wall 201 can also be designed to form multiple drug cavities. That is, the drug delivery part 2B includes the air cavity 2020 and multiple drug solution cavities 2010 which are mutually isolated. The air cavity 2020 is connected with the channel 10. The drug solution cavities 2010 are isolated from each other and they are located around the air cavity 2020 (between the inner wall 202 and the outer wall 201).

The through hole 2021 is located near the neck 12 of the inner wall 202. The through hole 2021 penetrates the wall of the inner wall 202, so that the air cavity 2020 is connected with the drug solution cavity 2010 to allow the liquid to enter the air cavity 2020 from the drug solution cavity 2010. The through hole 2021 includes two ends, in which the air end 2021A connecting the air cavity 2020 is lower than the liquid end 2021B connecting the drug solution cavity 2010. That is, the air end 2021A is closer to the holding part 1. Such an inclined through hole 2021 allows the liquid located in the drug solution cavity 2010 to enter the air cavity 2020 under the action of negative pressure (refer to FIG. 7B). As a preferred embodiment, the diameter of the through hole 2021 is 0.1˜0.4 mm, preferably 0.2˜0.3 mm. The diameter of the through hole 2021 should be selected according to the flow performance of the drug solution. If the viscosity of the drug solution increases and the fluidity becomes weak, the diameter should be increased as long as the following two conditions are met:

under the action of negative pressure, the drug solution can enter the air cavity 2020, and enter the channel 10 along the air cavity 2020, and then enter the micro-particles 4 in the channel 10 under the action of negative pressure; also in the absence of negative pressure (i.e. under normal pressure), the drug solution will not enter the air cavity 2020 (for example, the drug solution's surface tension prevent the itself enter the air cavity).

The medical micro-particles storage tube in the embodiment of the invention can be provided with only one drug solution cavity or multiple drug solution cavities 2010. Each drug solution cavity is connected with the through hole 2021. In practical use, different drug solutions can be injected into different drug cavities 2010. On the premise of meeting the compatibility requirements of pharmaceutics, different drug solutions are sucked into channel 10 by using negative pressure to load drugs in micro-particles.

The medical micro-particles storage tube in the embodiment of the invention also includes at least one adjusting cavity 2010′ (as shown in FIG. 7a), which is used to adjust the volume of the drug solution cavity. The adjusting cavity 2010′ has no through hole 2021 and is an individual chamber. The volume of the drug solution cavity or multiple drug solution cavities is adjustable by he adjusting cavity 2010′, so as to avoid the small amount of drug solution (such as 1 ml or a few microliters) being dispersed into the too large drug solution cavity and unable to be rapidly sucked into the through hole and the channel.

Since the air and the drug individually flow in isolated paths in the embodiment, the drug solution does not block the way of the air travels, so it will not generate bubbles, and thus avoid the splashing of the drug solution caused by the explosion of bubbles (which will waste drug solution). Moreover, without the large pressure difference as in the first embodiment (the pressure needs as large as to pump the air out from the drug solution), a low-power vacuum pump can be used to pump the air at low power and low speed in the present embodiment. Moreover, the drug loading capacity of the present embodiment is higher than that of the first embodiment.

### Fourth Embodiment

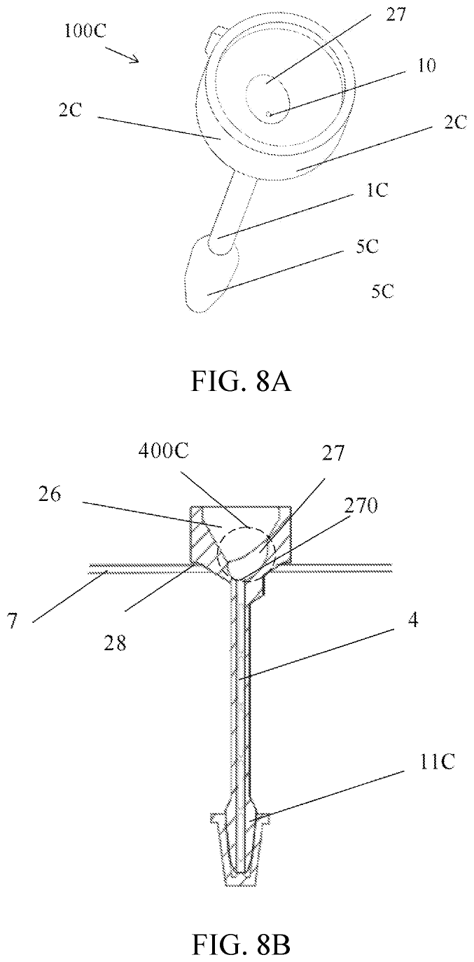

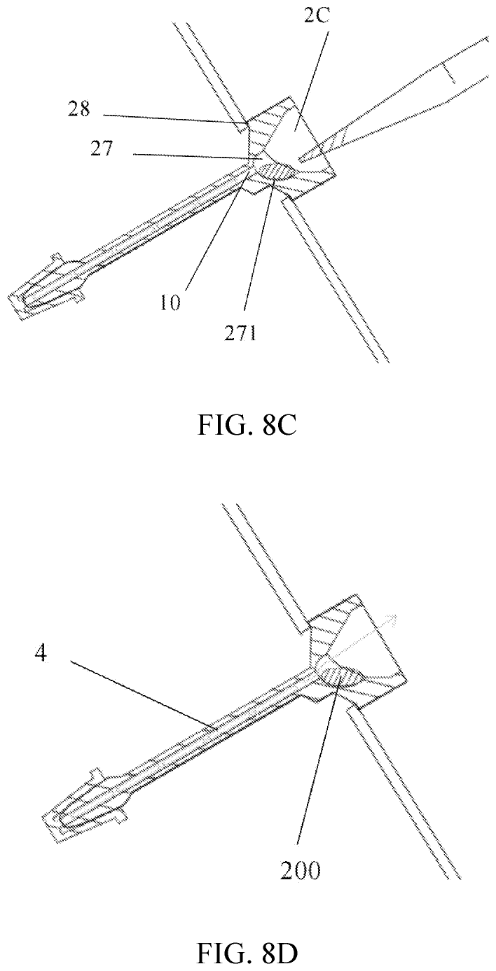

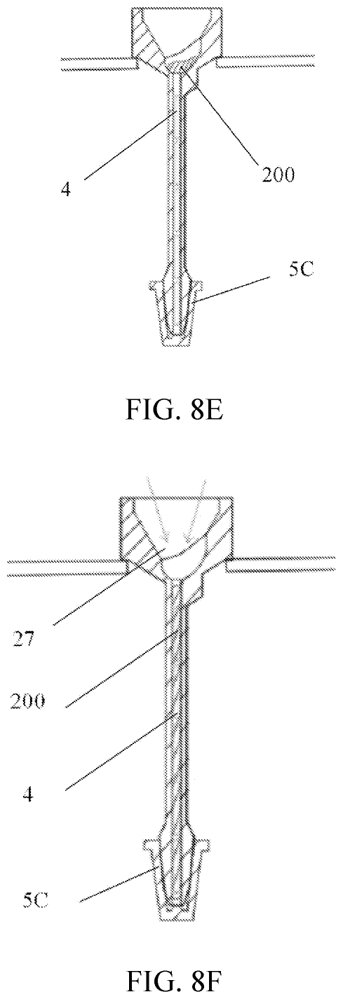

As shown in FIG. 8A to FIG. 8F, in the fourth embodiment of the present invention, the medical micro-particles storage tube 100C includes a holding part 1C and a drug delivery part 2C.

In this embodiment, the drug delivery part 2C includes a liquid collecting structure 400C for storing the drug solution 200. The liquid collecting structure 400C is designed to meet the following two conditions: 1) when the air does not exhaust, the liquid collecting structure 400C can store the drug solution 200; and 2) when the air is discharged along the channel 10 in the direction from the opening part 11C to the drug delivery part 2C (that is, in FIG. 8D, it flows out in the direction of the arrow), it will not pass through the drug solution 200. In this embodiment, the liquid collecting structure 400C is a liquid collecting cavity 27.

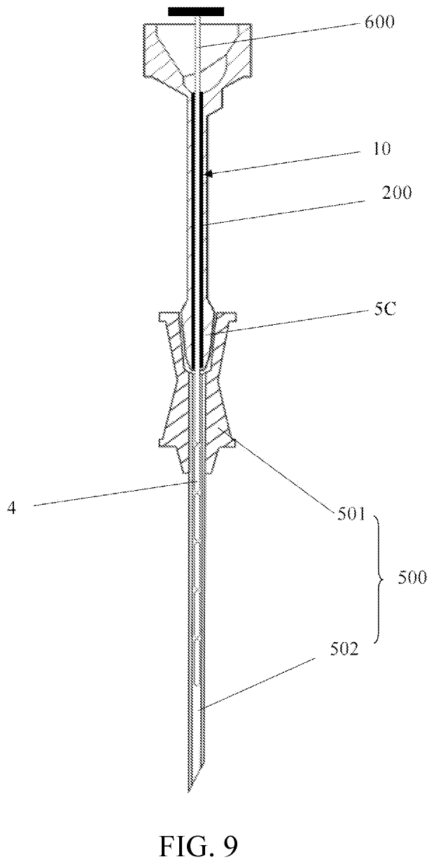

Similar to the first embodiment, the holding part 1C has a channel 10 extending along the axis of the hollow tube for delivering micro-particles 4. One end of the holding part 1C is connected with the drug delivery part 2C, and the other end is the opening part 11C, whose outer diameter is larger than other parts of the holding par 1Ct. The opening part 11C conforms to the Luer connector technical standard, such as ISO594-2:1998. As shown in FIG. 9, with the opening 11C, the medical micro-particles storage tube 100C can be matched with the Luer connector 501 arranged on the puncture needle 500 to ensure the reliable docking of the medical micro-particles storage tube and the puncture needle. In the docking state, the channel 10 of the medical micro-particles storage tube is aligned with the needle path 502 of the puncture needle 500, and the inner diameter of the channel 10 is less than or equal to the inner diameter of the needle path 502 of the puncture needle. It is well known that the puncture needle has a variety of specifications. Accordingly, the medical micro-particles storage tube provided by the embodiments of the present invention is also designed to have a variety of specifications, with different inner diameters.

The drug delivery part 2C includes a collecting part 26 and a liquid collecting part 27. The collecting part 26 has an irregular shape. The collecting part 26 is connected with the inner cavity of the liquid collecting part 27. The inner cavity size of the collecting part 26 is larger than that of the liquid collecting part 27, to collect the drug solution and transmit it to the liquid collecting part 27. The collecting part 26 of the present embodiment replaces the collecting part 21 and the gathering part 22 of the first embodiment, so that the drug solution from the collecting part 26 can converge to the liquid collecting part 27.

One end of the liquid collecting part 27 is connected with the collecting part 26, and the other end is connected with the channel 10. The shape of the liquid collecting part 27 is an eccentric tube structure relative to the axis of the channel 10. Specifically, when the liquid collecting part 27 is in a liquid delivery state, the drug delivery part 2C is in the upright state (see FIG. 8B). The lowest (or deepest) position of the liquid collecting part 27 in this state is called “the lowest position of liquid delivery 270”. In the liquid delivery state, the lowest position of liquid delivery 270 of the liquid collecting part 27 is located on/along the axis of the channel 10 (or near the axis). When the liquid collecting part 27 is in the liquid collecting state, the drug delivery part 2C is inclined (see FIG. 8C). The lowest part of the liquid collecting part 27 in the liquid collecting state is called “the lowest position of the liquid collecting 271”. The lowest position of the liquid collecting 271 is far away from the channel 10 and is different from the lowest position of liquid delivery 270.

Therefore, when the drug delivery part 2C is in the upright state, the liquid collecting part 27 is in the liquid delivery state, so that the liquid in the liquid collecting part 27 flows into the channel 10 from the lowest position of liquid delivery 270. When the drug delivery part 2C is inclined, the liquid collecting part 27 is in the liquid collection state, so that the drug solution 200 in the liquid collecting part 27 stays (accumulates) between the lowest position of liquid delivery 270 and the lowest position of the liquid collecting 271.

The outer surface of the drug delivery part 2C also forms a shoulder 28, which is used to cooperate with the support frame 7, so that the medical micro-particles storage tube 2C can also achieve mechanical balance in the inclined state to maintain the inclined state. As an alternative, the support frame 7 and the drug delivery part 2C can be one-piece structure. That is, the support frame 7 is fixed to the periphery of the drug delivery part 2C. In conclusion, the support frame can be fixed to or connected with the periphery of the drug delivery part 2C.

The drug loading method using the medical micro-particles storage tube 100C of the above embodiment is described below in combination with FIG. 8C to FIG. 8F.

As shown in FIG. 8C, after blocking the opening 11C, put at least one micro-particles 4 into the holding part 1C, tilt the medical micro-particles storage tube 100C (in this embodiment, tilt 45-70 degrees, preferably 60 degrees), and use the support frame 7 to maintain the medical micro-particles storage tube 100 inclined. The medical micro-particles storage tube 100C is tilted so that the lowest position of the liquid collecting 271 is below the axis of the channel 10 to allow the drug solution 200 to accumulate near the lowest position of the liquid collecting 271.

Then, a predetermined volume of drug solution 200 is injected. At this time, the drug delivery part 2C is inclined, the liquid collecting part 27 is in the liquid collection state, and the drug solution 200 can stay between the lowest position of the liquid delivery 270 and the lowest position of the liquid collection 271, so the drug solution 200 will stay in the liquid collecting part 27.

Next, as shown in FIG. 8D, while keeping the drug delivery part 2C in an inclined state by the support frame 7, negative pressure is applied to the holding part 1C to suck the air in the micro-particles 4 stored in the holding part 1C.

Then, as shown in FIG. 8E, turn the drug delivery part 2C to the upright state so that the lowest part of the liquid collecting part 27 is the lowest position of the liquid delivery 270. The drug solution 200 flows naturally to the lowest position of the liquid delivery 270 under the action of gravity. However, at this time, because the channel 10 is too thin, and the viscosity of different drug solutions 200 are different, under the effect of surface tension, only a small amount of drug solution 200 with high viscosity can flow into the channel 10. Therefore, most of the drug solution still stay in the liquid collecting part 27. Even the drug solution 200 with low viscosity will not all flow into the channel 10, and a small part of the drug solution still stays in the liquid collecting part 27.

Finally, as shown in FIG. 8F, pressurize the drug delivery part 2C so that all the drug solution 200 staying in the liquid collecting part 27 (shown in FIG. 8E) flows into the channel 10 under the pressure, and further enters the interior of the micro-particles 4 in the channel 10. At this time, all the drug solution 200 enters the channel 10.

Different from the first embodiment, the air in the channel 10 and the micro-particles 4 is pumped while the drug delivery part 2C maintaining inclined as is shown in FIG. 8D. In this state, the lowest position of the liquid collecting 271 is below the axis of the channel 10, so the drug solution 200 therein will not block the channel 10. Therefore, the air pumped out along the channel 10 mainly travels along the axis of the channel 10 and will flow out from above the drug solution 200 (as shown by the arrow in FIG. 8D). The moving direction of air does not pass through the drug solution 200 (that is, the drug solution does not block the moving direction of the air). So no bubble can be formed in the drug solution, and thus avoid the splashing of drug solution that is caused by the explosion of bubbles in the drug solution (which wastes drug solution). More importantly, because the air does not need break liquid to allow the liquid 200 to enter the channel 10, the power of the vacuum pump can be reduced, the cost can be reduced, and the drug loading can be increased.

When the drug loading is completed, the micro-particles can be implanted into the body. As shown in FIG. 9, remove or open the plug 5C on the medical micro-particles storage tube 100C, then insert the open part 11C into the Luer connector 501 of the puncture needle 500. Secondly, use the push rod 600 to extend from the collecting part 26 of the medical micro-particles storage tube 100C into the channel 10. Thirdly, push the micro-particles 4 from the channel 10 into the needle path 502 of the puncture needle 500. After all micro-particles 4 enter the needle path 502 of the puncture needle, remove the medical micro-particles storage tube 100C from the puncture needle 500.

Finally, the micro-particles located in the puncture needle 500 can be implanted into the body according to the conventional operation.

The embodiment of the invention also provides a drug loading method using the aforementioned medical micro-particles storage tube, which at least includes the following steps:
the micro-particles 4 are inserted into the channel 10 in medical micro-particles storage tubes 100, 100A, 100b and 100C;
the drug solution 200 is injected into the drug delivery part 2;
negative pressure is applied to the medical micro-particles storage tubes 100, 100A, 100b, 100C until no air escapes, or apply negative pressure for a fixed duration, the air pressure being configured to pump out the air without the drug solution 200; and
negative pressure is stopped (or apply positive pressure instead), and the drug solution 200 is injected into the channel 10 under normal pressure or positive pressure until the drug solution 200 stops flowing.

The embodiment of the present invention also provides a method for delivering drug loadable micro-particles using the aforementioned medical micro-particles storage tube, which at least includes the following steps:
the micro-particles 4 are sent into the channel 10 of the medical micro-particles storage tubes 100, 100A, 100B, 100C;
the open parts 11 or 11C of the medical micro-particles storage tubes 100, 100A, 100B and 100C, are aligned with the puncture needle 500; and
a push rod 600 is inserted into channel 10 to push micro-particles 4 out of channel 10.

To sum up, the medical micro-particles storage tube provided by the embodiment of the invention can be used to store micro-particles as a new pharmaceutical packaging method. Moreover, it can be used as a drug loading container for micro-particles, and also as a channel to implant micro-particles into the body. Such a design with three functions together enables fine micro-particles (not loaded with drugs) to be placed in the medical micro-particles storage tube in the biological sterile production workshop (GMP purification workshop). From drug loading to micro-particles implanting, the micro-particles are all kept in the same medical micro-particles storage tube, so as to prevent micro-particles from contamination.

Furthermore, combined with the tabletop drug loading device provided by the applicant's Chinese patent application CN202210197666.5, the drug can be loaded by doctors himself/herself, and then implanted into animals or human bodies in a short time. That is, the medical micro-particles storage tube according to the embodiments of the present invention can realize drug loading and implantation in the operating room, which is also conducive to avoiding micro-particles contamination.

More importantly, the medical micro-particles storage tube provided by the embodiment of the invention can help the doctor to be free of drug dispensing, allowing the doctor to load different drug solutions and implant the micro-particles according to the condition of each patient, so as to actually realize the customized precise treatment of one drug for one person.

It should be noted that each embodiment or deformation example of the invention is described in a relevant way. The same and similar parts between each embodiment or deformation example can be referred to each other. Each embodiment or deformation example focuses on the differences from other embodiments, but they are all realized based on the working principle of the medical micro-particles storage tube, so there is no need to repeat them here.

Several embodiments of the present invention have been described in detail above. For those skilled in the art, any obvious changes made to the invention without departing from the essence of the invention will constitute an infringement of the patent right of the invention and will bear corresponding legal liabilities.

1. A medical micro-particles storage tube (100) for implanting micro-particles (4) into a body in cooperation with a puncture needle (500), the medical micro-particles storage tube (100) comprising a holding part (1) and a drug delivery part (2), wherein
the holding part (1) is an elongated hollow tube with a channel (10) for accommodating the micro-particles (4); one end of the holding part (1) is connected with the drug delivery part (2), and the other end of the holding part (1) is an opening part (11) for discharging the micro-particles out of the medical micro-particles storage tube (100),
an inner diameter of the channel (10) is 1.0-1.8 times the inner diameter of a needle path (502) of the puncture needle (500);
an inner cavity (20) of the drug delivery part (2) is connected with the channel (10) for conveying drug solution (200) to the channel (10).

2. The medical micro-particles storage tube according to claim 1, wherein:
the drug delivery part (2) includes a liquid collecting structure (400, 400C) for accumulating the drug solution (200), and the liquid collecting structure (400, 400C) is configured to: 1) accumulate the drug solution (200) when no air exhaust; 2) ensure the air will not pass through the drug solution (200) when the air flows from the opening part (11) to the drug delivery part (2) along the channel (10); 3) allow the drug solution (200) can enter the channel (10) when the air flows from the drug delivery part (2) to the opening part (11).

3. The medical micro-particles storage tube according to claim 1, wherein:
the drug delivery part (2) comprises a collecting part (26) and a liquid collecting part (27),
one end of the liquid collecting part (27) is connected with the collecting part (26), and the other end is connected with the channel (10) for storing the drug collecting liquid (200).

4. The medical micro-particles storage tube according to claim 3, wherein:
the liquid collecting part (27) has a shape configured to meet the following requirements:
when the liquid collecting part (27) is in a liquid delivery state, the lowest part of the liquid collecting part (27) is located on the axis of the channel (10) as the lowest position of the liquid delivery (270);
when the liquid collecting part (27) is in a liquid collecting state, the lowest part of the liquid collecting part (27) is far away from the channel (10) as the lowest position of the liquid collecting part (271);
the lowest position of the liquid collecting part (271) is different from the lowest position of the liquid delivery (270).

5. The medical micro-particles storage tube according to claim 4, further comprising:
a support frame (7) fixed to or connected with a periphery of the drug delivery part (2) to maintain the medical micro-particles storage tube (100C) in an inclined state.

6. The medical micro-particles storage tube according to claim 1, wherein:
the opening part (11C) is configured to conform TO Luer interface technical standard.

7. The medical micro-particles storage tube according to claim 1, further comprising:
a plug (5) is detachably installed on the opening part (11) for plugging the opening parts (11, 11C).

8. The medical micro-particles storage tube according to claim 1, wherein:
the drug delivery part (2B) comprises an outer wall (201) and an inner wall (202),
the inner wall (202) forms an air cavity (2020), and the air cavity (2020) is aligned with the channel (10) to form an air path;
the inner wall (202) is connected with the outer wall (201) to form a drug solution cavity (2010) separated from the air cavity (2020);
the drug solution cavity (2010) is communicated with the air cavity (2020) through a through hole (2021).

9. The medical micro-particles storage tube according to claim 8, wherein:
a plurality of drug solution cavities (2010) is formed between the inner wall (202) and the outer wall (201).

10. The medical micro-particles storage tube according to claim 8, wherein:
the through hole (2021) comprises an air end (2021A) connecting the air cavity (2020) and a drug solution end (2021B) connecting the drug solution cavity (2010),
The air end (2021A) is closer to the channel (10) than the liquid end (2021B).

11. The medical micro-particles storage tube according to claim 10, wherein:
the through hole (2021) has a diameter that ranges from 0.1 to 0.4 mm.

12. The medical micro-particles storage tube according to claim 1, wherein:
the drug delivery part (2) comprises a collecting part (21) and a transition part (23);
one end of the transition part (23) is connected to the channel (10), and the other end is connected to the collecting part (21), and the inner diameter of the transition part (23) increases gradually from a diameter that is the same as that of the channel (10), to a diameter that is the same as that of the collecting part (21).

13. The medical micro-particles storage tube according to claim 1, wherein:
the drug delivery part (2) includes a collecting part (21), a transition part (23) and a gathering part (22), and the gathering part (22) connects the collecting part (21) and the transition part (23);
the collecting part (22) has an inner diameter which increases from a diameter that is the same as that of the transition part (23), gradually to a diameter that is the same as that of the collecting part (21);
the transition part (23) has an inner diameter which increases from a diameter that is the same as that of the channel (10), gradually to a diameter that is the same as that of the gathering part (22).

14. A drug loading method using the medical micro-particles storage tube (100, 100A, 100b, 100C) according to claim 1, comprising the following steps:
micro-particles (4) are inserted into a channel (10) in the medical micro-particles storage tube (100, 100A, 100b, 100C);
drug solution (200) is injected into a drug delivery part (2) of the medical micro-particles storage tube (100, 100A, 100b, 100C);
negative pressure is applied to the medical micro-particles storage tube (100, 100A, 100b, 100C) until no air escapes, or apply negative pressure for a fixed duration, with a negative pressure that allows the air to be pumped out without the drug solution (200);
the negative pressure is stopped, or positive pressure is applied, and then the drug solution (200) is injected into the channel (10) until the drug solution (200) stops flowing.

15. A method for delivering drug loadable micro-particles using the medical micro-particles storage tube according to claim 1, comprising the following steps:
micro-particles (4) are inserted into a channel (10) of the medical micro-particles storage tube (100, 100A, 100b, 100C);
an open part (11, 11C) of the medical micro-particles storage tube (100, 100A, 100b, 100C) is aligned with a puncture needle (500);
a push rod (600) is pushed into the channel (10), and the micro-particles (4) are pushed out of the channel (10).
